(19) United States
(12) Patent Application Publication
(10) Pub. No.: US 2007/0185409 A1
(43) Pub. Date: Aug. 9, 2007

(54) METHOD AND SYSTEM FOR DETERMINING AN OPERABLE STIMULUS INTENSITY FOR NERVE CONDUCTION TESTING

(76) Inventors: Jianping WU, Mississauga, ON (CA); Ronald Leon KURTZ, Oakville, ON (CA)

Correspondence Address: BERESKIN AND PARR, 40 KING STREET WEST, BOX 401, TORONTO, ON, M5H 3Y2, CA

(21) Appl. No.: 11/675,351

(22) Filed: Feb. 15, 2007

Related U.S. Application Data
(63) Continuation-in-part of application No. 11/557,390, filed on Nov. 7, 2006.
Application No. 11/557,390 is a continuation-in-part of application No. 11/407,296, filed on Apr. 20, 2006.
(60) Provisional application No. 60/774,646, filed on Feb. 21, 2006.
(60) Provisional application No. 60/774,646, filed on Feb. 21, 2006.
(60) Provisional application No. 60/672,853, filed on Apr. 20, 2005.

(51) Int. Cl. A61B 5/05; A61B 5/04

(52) U.S. Cl. 600/554; 600/546

(57) ABSTRACT

The described embodiments relate generally to methods, systems and apparatus for automatic nerve stimulation and for determining an operable stimulus intensity for nerve conduction testing. The method of determining an operable stimulus intensity for nerve conduction testing comprises: repeatedly stimulating a body portion adjacent a nerve at an increasing stimulus intensity; detecting a response potential in response to each stimulation of the body portion; determining a plurality of averaged responses based on the detected response potentials, each averaged response being an average of a set of at least two consecutive response potentials, each set of response potentials having at least one response potential not in another set; determining at least two parameters of each averaged response; determining that a maximal stimulus intensity has been reached when the respective at least two parameters of at least two averaged responses are within a predetermined percentage range; and determining the operable stimulus intensity as a predetermined proportion of the maximal stimulus intensity.

### CROSS-REFERENCE TO RELATED APPLICATIONS

This application claims the benefit of U.S. Provisional Patent Application Ser. No. 60/774,646, filed Feb. 21, 2006 and is a continuation-in-part of U.S. patent application Ser. No. 11/557,390, filed Nov. 7, 2006, which is a continuation-in-part of U.S. patent application Ser. No. 11/407,296, filed Apr. 20, 2006, which claims the benefit of U.S. Provisional Patent Application Ser. No. 60/774,646, filed Feb. 21, 2006 and the benefit of U.S. Provisional Patent Application Ser. No. 60/672,853 filed Apr. 20, 2005, the entire contents of all of which are hereby incorporated by reference.

### TECHNICAL FIELD

The described embodiments relate generally to methods, systems and apparatus for automatic nerve stimulation and for determining an operable stimulus intensity for nerve conduction testing. Embodiments of the invention may be used for conducting testing for Carpal Tunnel Syndrome, or other forms of systemic or entrapment neuropathies, for example.

### BACKGROUND

When performing nerve conduction testing, such as for Carpal Tunnel Syndrome, for example, a series of stimuli are provided to a part of the body adjacent to the nerve desired to be tested and the response of the body to each stimulus is measured. Such responses usually include a muscle response, in the form a compound muscle action potential (CMAP), and a nerve response, in the form of a sensory nerve action potential (SNAP). When performing the nerve conduction testing, it is desirable to provide the stimulus at a stimulus intensity that triggers a maximal or near maximal response. Depending on the physiological features of the body part and the particular person being tested, determining the optimal stimulus intensity for obtaining the maximal response can be problematic. In particular, manual methods for determining the optimal stimulus intensity can be relatively time consuming and cumbersome. One example of such manual methods is described in Kimura, J. Electrodiagnosis in Diseases of Nerve and Muscle: Principles and Practice, Oxford University Press, U.S.A., 2001, 3^{rd} Edition, p. 97.

Some methods for determining the optimal stimulus intensity rely on measuring the peak amplitude of the response. As the peak amplitude may fluctuate depending on a number of conditions, this can be a somewhat unreliable basis on which to determine whether the maximal stimulus intensity has been reached. If only the peak amplitude of the response is used in determining the maximal stimulus intensity, responses having spuriously low or high peak amplitudes may result in a false determination of the maximal stimulus intensity.

It is desired to address or ameliorate one or more of the problems or shortcomings associated with previous methods, systems and apparatus, or to at least provide a useful alternative thereto.

### SUMMARY

The described embodiments relate generally to methods, systems and apparatus for automatic nerve stimulation and for determining an operable stimulus intensity for nerve conduction testing. Embodiments of the invention may be used for conducting testing for Carpal Tunnel Syndrome, or other forms of systemic or entrapment neuropathies, for example.

Certain embodiments relate to a method of determining an operable stimulus intensity for nerve conduction testing. The method comprises repeatedly stimulating a body portion adjacent a nerve at an increasing stimulus intensity; detecting a response potential in response to each stimulation of the body portion; determining a plurality of averaged responses based on the detected response potentials, each averaged response being an average of a set of at least two consecutive response potentials, each set of response potentials having at least one response potential not in another set; determining at least two parameters of each averaged response; determining that a maximal stimulus intensity has been reached when the respective at least two parameters of at least two averaged responses are within a predetermined percentage range; and determining the operable stimulus intensity as a predetermined proportion of the maximal stimulus intensity.

The predetermined proportion may be 110%. The predetermined percentage range may be 0 to 20%. The at least two averaged responses may comprise three averaged responses. The at least two parameters may be selected from the group consisting of: onset time, peak amplitude and the area of the response potential between peak amplitude and onset. In one embodiment, each averaged response comprises an average of three consecutive response potentials.

The steps of stimulating and detecting may comprise: a) determining a stimulus intensity at which to provide a stimulus to the body portion; b) stimulating the body portion with the stimulus at the stimulus intensity; c) detecting the response potential of the body portion in response to the stimulus; d) reducing the stimulus intensity by a first predetermined amount and once repeating steps b) and c); e) verifying that the reduced stimulus intensity corresponds to detecting a reduced response potential in response to the reduced stimulus intensity; f) increasing the stimulus intensity by a second predetermined amount larger than the first predetermined amount and once repeating steps b) and c); and g) repeating steps d), e), and f) until the detected response potential is determined to be a maximal response potential.

Other embodiments relate to a method of automatic nerve stimulus. The method comprises: a) determining a stimulus intensity at which to provide a stimulus to a body portion adjacent a nerve; b) stimulating the body portion with the stimulus at the stimulus intensity; c) detecting a response potential of the body portion in response to the stimulus; d) reducing the stimulus intensity by a first predetermined amount and once repeating steps b) and c); e) verifying that the reduced stimulus intensity corresponds to detecting a reduced response potential in response to the reduced stimulus intensity; f) increasing the stimulus intensity by a second predetermined amount larger than the first predetermined amount and once repeating steps b) and c); and g) repeating steps d), e), and f) until the detected response potential is determined to be a maximal response potential.

Step f) may further comprise verifying that the increased stimulus intensity corresponds to detecting an increased response potential in response to the increased stimulus intensity.

Further embodiments relate to computer readable storage storing computer program instructions which, when executed by a computerized testing apparatus, cause the computerized testing apparatus to perform the methods described above.

Still other embodiments relate to a system for determining an operable stimulus intensity for nerve conduction testing. The system comprises: a stimulator for stimulating a body portion adjacent a nerve and detecting a response to stimulation of the body portion; a control module for controlling the stimulator; and memory storing program instructions and accessible by the control module. When the program instructions are executed by the control module, the control module and stimulator are caused to: repeatedly stimulate the body portion at an increasing stimulus intensity; detect a response potential in response to each stimulation of the body portion; determine a plurality of averaged responses based on the detected response potentials, each averaged response being an average of a set of at least two consecutive response potentials, each set of response potentials having at least one response potential not in another set; determine at least two parameters of each averaged response; determine that a maximal stimulus intensity has been reached when the respective at least two parameters of at least two averaged responses are within a predetermined percentage range; and determine the operable stimulus intensity as a predetermined proportion of the maximal stimulus intensity.

Still further embodiments relate to a system for automatic nerve stimulus. The system comprises: a stimulator for stimulating a body portion adjacent a nerve and detecting a response to stimulation of the body part; a control module for controlling the stimulator; and memory storing program instructions and accessible by the control module. When the program instructions are executed by the control module, the control module and stimulator are caused to: a) determine a stimulus intensity at which to provide a stimulus to the body portion; b) stimulate the body portion with the stimulus at the stimulus intensity; c) detect a response potential of the body portion in response to the stimulus; d) reduce the stimulus intensity by a first predetermined amount and repeat steps b) and c); e) verify that the reduced stimulus intensity corresponds to detecting a reduced response potential in response to the reduced stimulus intensity; f) increase the stimulus intensity by a second predetermined amount larger than the first predetermined amount and repeat steps b) and c); and g) repeat steps d), e), and f) until the detected response potential is determined to be a maximal response potential.

### BRIEF DISCRIPTION OF THE DRAWINGS

Embodiments of the invention are described in further detail below, by way of example only, with reference to the accompanying drawings, in which:

FIG. 1 is a block diagram of a system for automatic nerve conduction testing;

FIG. 2 is a diagram illustrating connection of a coupling unit to a stimulus unit when the stimulus unit is attached to a wrist and hand area on an arm;

FIG. 3 is a perspective view of the coupling unit and stimulus unit of FIG. 2, shown connected and showing insertion of a distance measurement member into the coupling unit;

FIG. 4 is a schematic representation of a stimulus unit according to one embodiment;

FIG. 5 is a schematic representation of a stimulus unit according to another embodiment;

FIG. 6 is a block diagram illustrating an exemplary data flow for automatic nerve conduction testing;

FIG. 7 is an example plot of a CMAP evoked response, showing amplitude variations over time;

FIG. 8 is an illustrative graph of stimulus intensity over time when performing a method of determining an operable stimulus intensity; and

FIG. 9 is a flow chart of a method of administering stimuli to a body part.

### DETAILED DESCRIPTION

Embodiments of the invention can be used to apply an automatic nerve conduction test for systemic or entrapment neuropathies, for example such as Carpal Tunnel Syndrome. During the test, a series of impulse stimuli are applied to a subject's body part adjacent a nerve or nerve group. The responses to the stimuli are analyzed to detect the evoked action potentials (CMAP, for a motor nerve test, and SNAP for a sensory nerve test), and to measure the onset latency and peak amplitude of the responses. In order to obtain the most meaningful measurements, it is necessary to ensure that the evoked responses being measured for diagnostic proposes are maximal responses. To obtain maximal responses, a corresponding maximal stimulus must be applied to the nerve or nerve group. Thus, prior to the diagnostic testing, it is necessary to determine the maximal stimulus intensity.

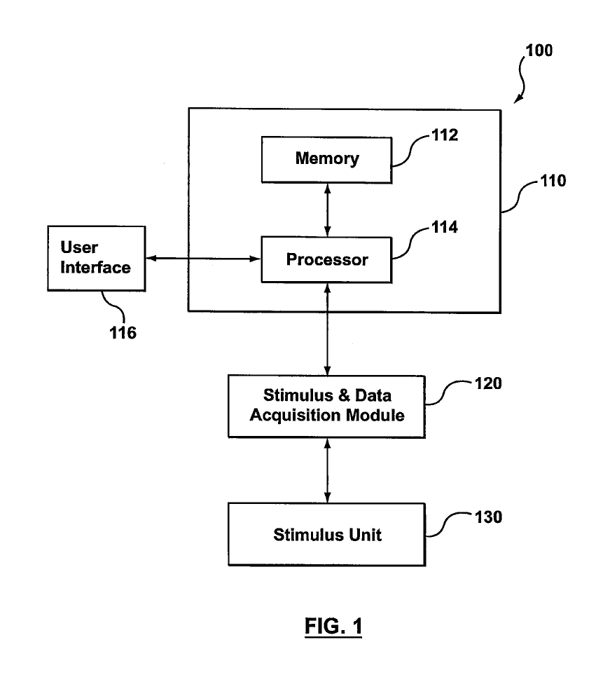

Referring to FIG. 1, there is shown a system 100 for performing automatic nerve conduction testing. System 100 comprises a control module 110 that interfaces with a stimulus unit 130 via a stimulus and data acquisition module 120 to provide stimuli to a body part and detect responses, such as CMAP and SNAP responses, to the stimuli. Other evoked responses that may be detected include F-wave, A-wave and H-reflex responses.

Control module 110 may be in the form of a computer device, such as a laptop, desktop personal computer or a handheld computing device. Control module 110 comprises a processor 114 and memory 112. Control module 110 has a user interface 116 associated therewith that communicates with processor 114 to enable a user to interface with system 100 during, before or after the testing. The memory 112 stores computer program instructions for execution by processor 114 during performance of the automatic nerve conduction testing. Memory 112 alos stores a first-in-first-out stack of sampled response waveforms (traces) for analysis by processor 114. Processor 114 controls stimulus and data acquisition module 120, which in turn controls the output of stimulus unit 130.

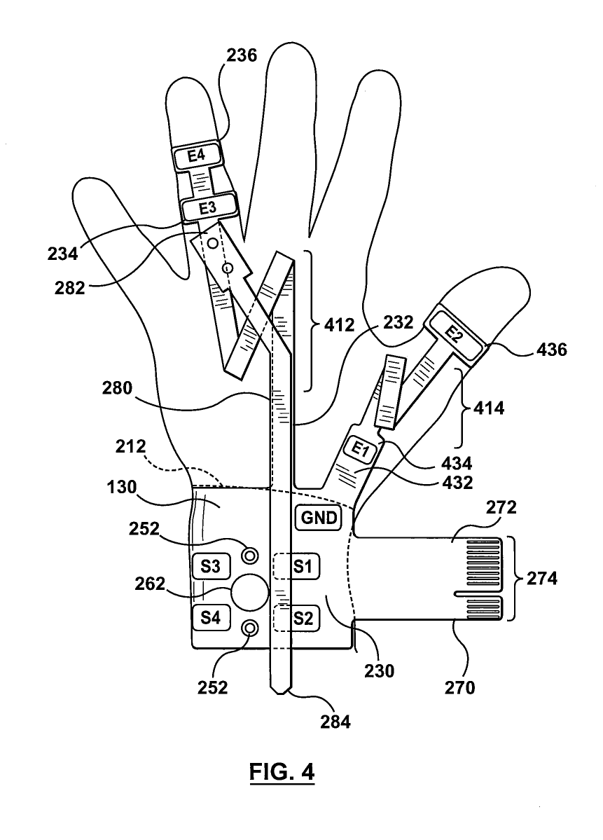

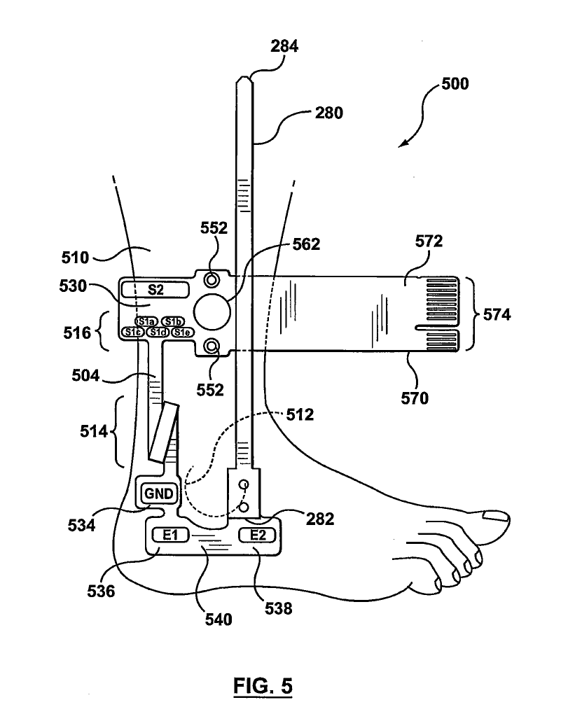

Stimulus unit 130 has one or more stimulus electrodes (for example, S1, S2, S3 and S4, shown in FIG. 4) for contacting the skin of the body adjacent a nerve that is desired to be tested and also has one or more sensing electrodes (for example, E1, E2, E3 and E4, shown in FIG. 4) for sensing the action potentials, such as CMAP and SNAP, on the skin at body part locations spaced from the stimulus sites. According to one embodiment, the stimulus unit 130 may be used to stimulate more than one nerve grouping at the same time. For example, stimulus unit 130 may be used to stimulate the median and ulnar nerve groupings in the hand simultaneously and separately detect the responses to that stimulation. Alternatively, stimulus unit 130 may detect responses to stimulus of only a single nerve grouping. Examples of such embodiments of stimulus unit 130 are shown in FIGS. 4 and 5 and described below. Further examples of suitable stimulus units are shown and described in U.S. patent application Ser. No. 11/557,390.

Stimulus and data acquisition module 120 has one or more controllers (not shown) for receiving and interpreting commands from processor 114, for conditioning response signals received from stimulus unit 130 and providing such conditioned response signals to processor 114 for analysis according to the stored computer program instructions in memory 112. Example commands received at stimulus and data acquisition module 120 from processor 115 include stimulus intensity setting commands and operational commands such as start or stop commands. Additionally, if stimulus unit 130 is configured to provide (or cooperate with stimulus and data acquisition module 120 to provide) a temperature measurement or a measurement of the distance between the stimulation and detection points, such measurements may be provided to processor 114 in response to an appropriate command received at stimulus and data acquisition module 120.

The task of processor 114 is to analyze each stimulus-response waveform passed from the signal detection and processing framework (i.e. stimulus unit 130 and stimulus and data acquisition module 120) and to determine the next stimulus intensity according to the current and historical results until the desired number of supra-maximal responses is acquired and measured.

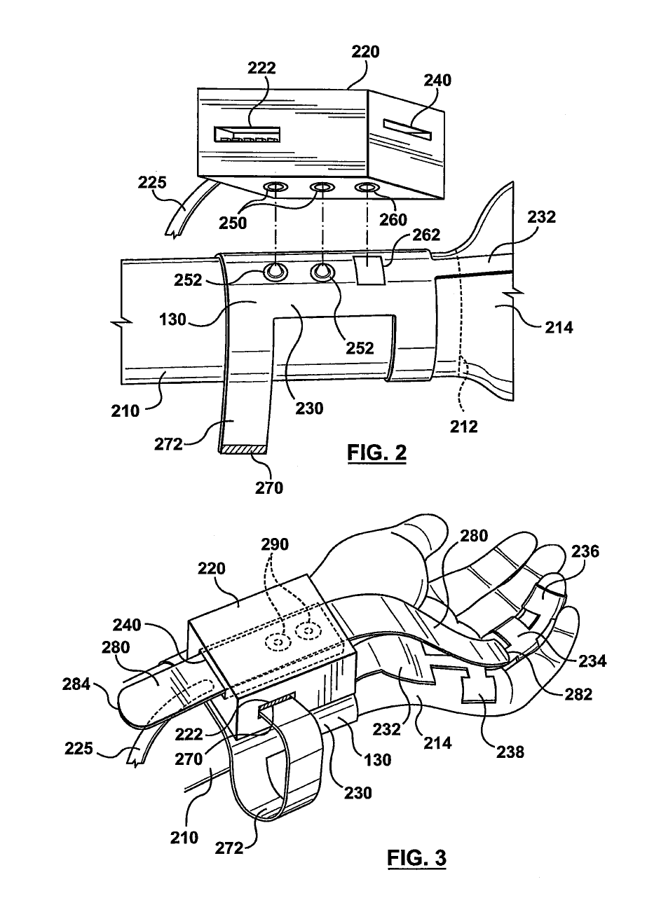

Referring also to FIGS. 2 and 3, stimulus unit 130 is shown in further detail, in use on a wrist and hand area of a person's arm. Stimulus unit 130 connects electrically with stimulus and data acquisition module 120 via a coupling unit 220, which couples directly to stimulus unit 130 to provide a stimulus current and to receive the evoked action potentials in response.

Coupling unit 220 forms part of stimulus and data acquisition module 120. Coupling unit 220 may be a dumb unit, in that it may not contain a controller or digital signal processor (DSP) exercising specific control or processing functions. In this case, another processor or controller inside stimulus and data acquisition module 120, located away from coupling unit 220 and in communication therewith via cable 225, performs the stimulation control and signal processing functions. Alternatively, coupling unit 220 may include a controller for performing stimulus control and/or received signal processing functions.

Coupling unit 220 couples to stimulus unit 130 by one or more mechanical connectors to position coupling unit 220 in a fixed location relative to stimulus unit 130. The connectors shown in FIG. 2 are snap connectors, with receiving parts 250 located on an underside of coupling unit 220 and projecting parts 252 located on an upper surface of stimulus unit 130. These connecting parts may be formed of conductive material, such as a conductive metal, for enabling a current stimulus to be provided from coupling unit 220 to stimulus unit 130 via the one or more connectors. Example conductive metals include nickel-plated brass or stainless steel. Instead of snap connectors, other forms of conductive connector may be employed.

In one embodiment, snap connector parts 250, 252 are not used for providing current stimulus signals, but are instead used to close a circuit (with a conductor extending between the two projecting parts 252) to provide an indication to stimulus and data acquisition module 120 that coupling unit 220 is connected to stimulus unit 130. In a further alternative embodiment, one or more non-conductive connecting parts may be used to form a connector connecting coupling unit 220 to stimulus unit 130.

Stimulus unit 130 has an output connector 270 located on an end of a connector limb 272 for providing evoked response signals detected by the one or more sensing electrodes to stimulus and data acquisition module 120, via coupling unit 220. Output connector 270 is releasably received in a socket 222 formed in coupling unit 220. Socket 222 has a structure formed for receipt of output connector 270 and for forming electrical connections with each of the conductors (which are, in turn, connected to the stimulus and/or sensing electrodes) along connector limb 272. Connector limb 272 resembles a flexible ribbon cable. If the current stimulus wave-forms are not provided to stimulus unit 130 by the physical connection of connecting parts 250, 252, then they may be provided by conductors connected to the stimulating electrodes via connector 270.

Stimulus unit 130 has a base portion 230, with at least one limb 232 extending therefrom, in addition to connector limb 272. Limb 232 has at least one sensing electrode positioned on the limb 232 for placement at any desired site for detection of CMAP or SNAP (or both) responses, depending on the type of testing that is to be conducted. One or more stimulus electrodes, together with a ground electrode (GND), are located in or adjacent base portion 230. Limb 232 extends distally of wrist crease 212 and crosses at least part of the palm 214. As shown in FIG. 3, limb 232 has two sensing electrodes 234, 236 located toward a distal end of limb 232. Optionally, a third sensing electrode 238 may be located more proximally on limb 232, intermediate base portion 230 and distal sensing electrodes 234, 236, for sensing a CMAP response from the hypothenar area.

Stimulus unit 130 is formed mostly of flexible materials for placement on anatomical structures and for generally conforming to the shape of such anatomical structures. For example, base portion 230 is intended to be positioned proximally of a wrist crease 212 so is to extend at least partially along and around part of a forearm 210. Certain parts of stimulus unit 130 (for example, those around the electrodes) have an adhesive substance, such as a foam adhesive layer, on a underside thereof, for affixing the stimulus unit to the relevant anatomical structures prior to testing. Flexible circuitry extends through stimulus unit 130 between the electrodes and connectors. Thus, stimulus unit 130 can be used with anatomical structures of varying shapes and sizes due to its flexibility and adaptability to conform and adhere to anatomical structures, as required.

Stimulus unit 130 employs a substrate of a flexible material, such as a medical grade polyester film (or other materials having similar properties). The substrate may be about 3 to 8 thousandths of an inch thick, for example. Where adhesive is required to affix a part of the stimulus unit 130 to an anatomical structure, this adhesive may be provided on a layer of medical grade adhesive foam of about 1/32 of an inch thickness. The foam is adhered to an insulation layer on the substrate on one side with a relatively strong adhesive and has an adhesive of relatively less strength for removable attachment to the test subject. The electrodes may comprise a silver or silver chloride layer formed on the substrate. The substrate also has flexible circuit tracings formed thereon for constituting the conductors between electrodes and the input and/or output connector. Such circuit tracings may comprise silver and a dielectric layer. An example of the layers of stimulus unit 130 is shown and described in further detail in U.S. patent application Ser. No. 11/407,296.

Prior to affixation to the body part, stimulus unit 130 may have backing sheets on those part of stimulus unit 130 that have an adhesive substance on their undersides for adhesion to the skin. Each such backing sheet is removed immediately prior to adhesion of the relevant part of stimulus unit 130 to the corresponding anatomical structures. For the sensing, stimulus and ground electrodes, an area of conductive gel, such as hydrogel, is interposed between the electrode and the skin surface (instead of the adhesive foam), for facilitating conductivity of electrical signals between the electrodes and the skin.

Stimulus unit 130 is a generally flat device, as viewed from the user's perspective, prior to affixation to the test subject. However, stimulus unit 130 does have several layers, as described above. In use of stimulus unit 130, and with the backing sheets removed, the adhesive foam parts and electrodes are positioned to lie against the skin. These skin contact surfaces may be conveniently referred to as being formed on the underside of the stimulus unit 130. Printed labeling, including affixation instructions, may be provided on the side of stimulus unit 130 that does not contact the skin.

Coupling unit 220 has a temperature sensor 260, such as an infrared temperature sensor, positioned on a lower surface of coupling unit 220 that is to be positioned to face the body part when coupled to stimulus unit 130. Temperature sensor 260 is used to detect the temperature of the skin prior to and/or during the testing. If temperature sensor 260 is used to take a temperature measurement prior to initiation of the testing, it can be placed over the palmar region or other anatomical structure, as appropriate, prior to connection of coupling unit 220 to stimulus unit 130. Alternatively, the temperature measurement may be obtained after connection of coupling unit 220 to stimulus unit 130, provided that stimulus unit 130 has an appropriate opening 262 to allow temperature sensor 260 to directly sense the skin temperature.

Coupling unit 220 also has a slot 240 formed in a housing of coupling unit 220 for receiving a distance measurement strip 280. Slot 240 extends all the way through coupling unit 220 so that the distance measurement strip 280 can be drawn though slot 240 in order to perform the distance measurement function, as described herein. In the embodiment shown in FIG. 3, scanners 290, such as optical scanners, are used to scan indicia located on distance measurement strip 280 between a free end 284 and a fixed end 282, which is attached to a connection portion on limb 232 in the vicinity of a sensing electrode.

Fixed end 282 may be attached to limb 232 by an adhesive or a mechanical connection, for example. Fixed end 282 may be attached to limb 232 in such a way that allows the distance measurement strip to be manually torn off or otherwise removed once it has been used.

Example distance measurement strips having different forms of indicia are shown in U.S. patent application Ser. No. 11/407,296. For the embodiment shown in FIG. 3, the indicia on distance measurement strip 280 are optically readable indicia that can be read by scanners 290 as the distance measurement strip 280 and the indicia thereon passes by the scanners 290 when distance measurement strip 280 is drawn through slot 240 in coupling unit 220.

Scanners 290 are located within the housing of coupling unit 220 and are positioned to sense indicia on the distance measurement strip 280 and to provide output signals to stimulus and data acquisition module 120 via cable 225. The electrical signals corresponding to the scanned optical indicia are processed within stimulus and data acquisition module 120 to determine the distance between the stimulus electrodes, which are in a fixed position relative to optical scanners 290, and sensing electrodes located on a distal extremity of the body part, such as a finger, the size and length of which will depend on the physical characteristics of the test subject.

The distance measurement calculation is performed by stimulus and data acquisition module 120, taking into account the point along distance measurement strip 280 at which scanners 290 are positioned when distance measurement strip 280 is at rest within slot 240, the known distance between scanners 290 and the stimulating electrodes when coupling unit 220 is connected to stimulus unit 130 and the known distance between the point at which fixed end 282 is connected to limb 232 and the sensing electrodes 234, 236 located on limb 232.

Depending on the type and/or configuration of the indicia on distance measurements strip 280, only one scanner 290 may be necessary. For example, if the indicia comprise gray scale indications, only one optical scanner may be required. However, if the indicia comprise offset quadrature indicia, two scanners are required to be able to determine the distance based on such indicia. Alternatively, the pair of quadrature scanners 290 may be offset and the indicia aligned with no offset.

In alternative embodiments, indicia other than optically readable indicia may be formed in, positioned on or otherwise fixed in relation to distance measurement strip 280 for enabling determination of the distance between the sensing electrodes and stimulation electrodes. Mechanical markings or formations may be applied to distance measurement strip 280, for example, in the form of crenelations along one edge or deformations in part of the strip. Alternatively, electrical or magnetic indicia may be formed in, or in relation to, distance measurement strip 280 for sensing by corresponding sensors in coupling unit 220. Whether the indicia is optical, mechanical, electrical, magnetic, a combination of two or more of these or any other machine-readable form, the indicia are, at least according to such embodiments, configured to be read using an appropriate sensing means positioned within or on coupling unit 220 for generating electrical signals for transmission to a signal processor within stimulus and data acquisition module 120 via cable 225.

In other alternative embodiments, the distance measurement strip 280 may be provided with human readable indicia for alignment with a fixed visible alignment marker on coupling unit 220 or a part of base portion 230, so that a person may readily determine from the human readable indicia and the alignment marker the distance between the sensor electrodes and the stimulus electrodes. Alternatively, instead of distance measurement strip 280 being fixed at a location near the sensor electrodes and having its free end extend across base portion 230, distance measurement strip 280 may be fixed at a location on or adjacent base portion 230 and extending toward the sensing electrodes for alignment of human readable indicia on the strip with an alignment marker positioned at a particular location on limb 232 adjacent to the sensing electrodes. For such embodiments using human readable indicia, the distance measurement determined with reference to the alignment marker would need to be input into control module 110 via user interface 116.

In a further alternative embodiment using human readable indicia, coupling unit 220 may be provided with an extensible measuring strip that retractably extends from coupling unit 220 for visual comparison with an alignment marker positioned adjacent one or more of the SNAP sensing electrodes 234, 236. In an alternative of such an embodiment, the retractable strip may use machine-readable indicia to determine the distance according to indicia that can be read from the strip by a scanner within coupling unit 220 when a free end of the retractable strip is positioned at the alignment marker.

Particular embodiments of further optical distance measurement methods may include use of stereoscopic optical sensors, triangulation of a marker light (where the marker is attached at or adjacent the sensing electrodes and the optical sensor is located in the coupling unit 220) and optical pattern recognition techniques. In a further embodiment, an acoustic time-of-flight calculation may be performed in relation to a marker source attached at or adjacent the sensing electrodes, with the acoustic sensor located in the coupling unit 220. Embodiments employing electrical distance measurement may include sensing a deformation of a wire loop having a modified self-inductance depending on its position along the distance measurement strip or along an extensible section in limb 232.

Electromechanical embodiments may use transducers, such as strain gauges, potentiometers or linear variable differential transformers (LVDT). Such embodiments may use structure embedded within distance measurement strip 280 or an extensible section in limb 232 in combination with corresponding sensing structure and circuitry within coupling unit 220. Specific mechanical distance measurement embodiments may employ a form of tape measure built into coupling unit 220, with sensors to determine the position or rotation of the tape wheel within coupling unit 220 and/or human readable indicia visible on the tape as it is extended from the coupling unit 220.

In certain embodiments, stimulus unit 130 may be employed with only a simple mating connector to connect to connector 270 in place of coupling unit 220. For such an embodiment, as there is no necessity to connect coupling unit 220 to stimulus unit 130, connector projections 252 are not required. Also, without a temperature sensor 260, opening 262 in stimulus unit 130 is not required.

The embodiment of stimulus unit 130 shown in FIGS. 2 and 3 has a base portion 230, a connector limb 272 and a distally extending limb 232 connected to, and extending away from, the base portion 230. Connector limb 272 is connected to, and extends away from, a proximal part of base portion 230. The base portion 230 is used to position the stimulation electrodes adjacent the nerve bundle desired to be stimulated during the testing, while the limb 232 extends distally to position the sensing electrodes in the desired locations for sensing SNAP and/or CMAP evoked responses. The connector limb 272 is used to couple to the stimulus and data acquisition module 120 and provide output signals corresponding to the electrical signals coupled to the conductors exposed by connector 270.

The base portion 230, distally extending limb 232 and connector limb 272 form a basic configuration of the stimulus unit 130. Variations of such a basic configuration form further embodiments, as described below. For example, stimulus unit 130 may have more than one distally projecting limb 232. Further, connector limb 272 may extend from a different part of the base portion 230, depending on whether the stimulus unit is for right hand or left hand testing, for example. While the precise shape and configuration of base portion 230 may vary, the features and functions of base portion 230 according to the basic configuration described above are common to all embodiments.

Referring also now to FIG. 4, one particular embodiment of stimulus unit 130 is shown schematically, as located on a person's right hand for performing median and ulnar nerve conduction testing,

Base portion 230, as shown in FIG. 4, has two stimulation electrode pairs S1, S2 and S3, S4 formed in the substrate. The first stimulation electrode pair S1, S2 is to be positioned over the median nerve running centrally through the wrist, while the second electrode pair S3, S4 is to be positioned over the ulnar nerve. In the example shown in FIG. 4, a distal edge of base portion 230 is approximately aligned with the wrist crease 212 and the base portion 230 is fixed in position by adhesion with the skin. In this position, a ground electrode GND is positioned distally of the stimulation electrodes but proximally of the sensing electrodes and generally toward a distal edge or area of base portion 230.

Stimulus unit 130, as shown in FIG. 4, has a first limb 232 extending distally from base portion 230 for attachment to the fourth digit (ring finger) on the right hand. Fixed end 282 of distance measurement strip 280 is affixed to limb 232 at a connection portion adjacent, but proximal of, sensing electrode 234. Free end 284 of distance measurement strip 280 extends proximally from fixed end 282 for passing through slot 240, when coupling unit 220 is connected to the stimulus unit 130.

Stimulus unit 130, as shown in FIG. 4, has a second limb 432 connected to, and extending distally from, base portion 230. Second limb 432 has first and second sensing electrodes E1, E2 formed in respective first and second attachment portions 434, 436 having adhesive on an underside thereof for holding the sensing electrodes E1, E2 on to the skin at desired locations. Sensing electrode E1 is positioned approximately over the middle of the thenar area, while sensing electrode E2 is wrapped around a distal joint of the thumb.

The first and second limbs 232, 432 each have a respective extensible portion 412, 414 for accommodating size differences among hands by allowing lesser or greater extension of the extensible portions 412, 414, depending on hand size. Extensible portions 412, 414 may be formed of a somewhat flattened coil or loop in the respective limb.

The stimulus unit 130 shown in FIG. 4 has a connector 270 with a plurality of connecting conductors 274 located at an end of connector limb 272. Connecting conductors 274 communicate with conductors formed in the substrate of stimulus unit 130 and extending through the limbs 232, 432 and base portion 230. Connecting conductors 274 connect with corresponding conductors in socket 222 of coupling unit 220.

Referring now to FIG. 5, there is shown a further embodiment of a stimulus unit, designated by reference numeral 500. Stimulus unit 500 is intended for use in nerve conduction testing of the sural nerve in a human leg. Stimulus unit 500 has a base portion 530 for location over the sural nerve on a lower part of a right leg, as shown in FIG. 5. Coupling unit 220 is usable with stimulus unit 500 in a similar manner to that described with reference to FIGS. 2 and 3.

Connected to base portion 530 is a connector limb 572 having a connector 570 on an end thereof and connector conductors 574 exposed within connector 570. Connector 570 is receivable in the socket 222 of coupling unit 220 in a manner similar to that described in relation to connector 270. Similar to base portion 230, base portion 530 has snap projections 552 for connecting to corresponding recesses in coupling unit 220.

Base portion 530 has a reference stimulation electrode S2 formed on the substrate and an array 516 of active stimulation electrodes (S1a, S1b, S1c, S1d, S1e) formed distally of S2 on the substrate. The array 516 is used to selectively provide stimuli to different locations within a stimulus area covered by the array 516.

The substrate of stimulus unit 500 further comprises a distally extending limb 504 connected to, and integrally formed with, base portion 530. Limb 504 has an extensible portion 514 formed therein for allowing adjustment of the distance between the sensing and stimulus electrodes to account for different leg sizes. A distal end portion 540 is formed at a distal end of limb 504 and comprises sensing electrodes E1, E2. A ground electrode GND is also formed in limb 504, intermediate distal end portion 540 and the extensible portion 514.

Distal end portion 540 has adhesive attachment portions 536, 538 for securing electrodes E1, E2 to the skin of the ankle just below, and on either side of, the lateral malleolus 512. Ground electrode GND is attached to the skin using an adhesive attachment portion 534.

Distance measurement strip 280 is connected at fixed end 282 to a part of distal end portion 540 adjacent attachment portion 538. Distance measurement strip 280 extends proximally toward base portion 530 so that free end 284 can be passed through slot 240 of coupling unit 220 for measurement of the distance between the sensing electrodes E1, E2 and the stimulation electrodes S2, S1a to S1e.

As shown in FIG. 5, opening 562 in base portion 530 is located between projecting connector parts 552. For such a configuration, the coupling unit 220 may have a temperature sensor 260 positioned in between recessed connecting parts 250 to correspond with the configuration of base portion 530. Such a modified coupling unit 220 may also be used with the stimulus unit shown in FIG. 4, with opening 262 being positioned in between projecting connector parts 252.

It should be noted that stimulus unit 500 is one specific embodiment of the more general embodiment of stimulus unit 130 described above. Thus, while stimulus unit 500 is of a different shape and configuration to that shown in FIGS. 3 and 4, for example, it is formed in a similar manner, using similar materials and is used in a similar way.

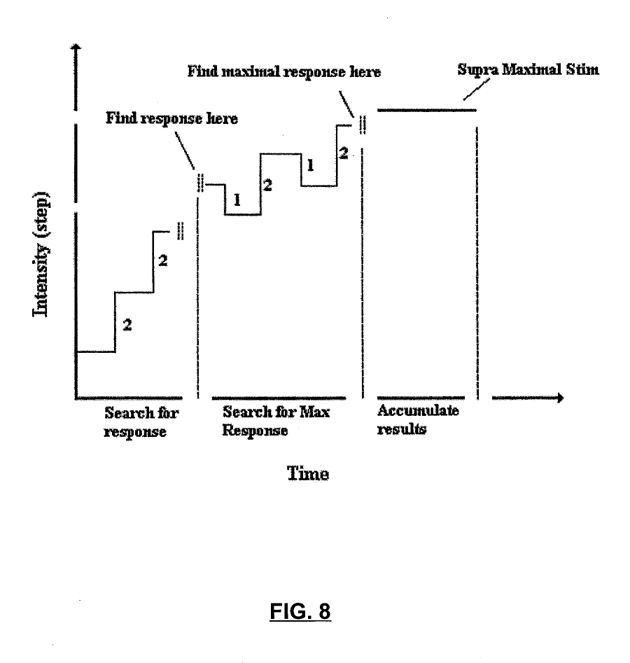

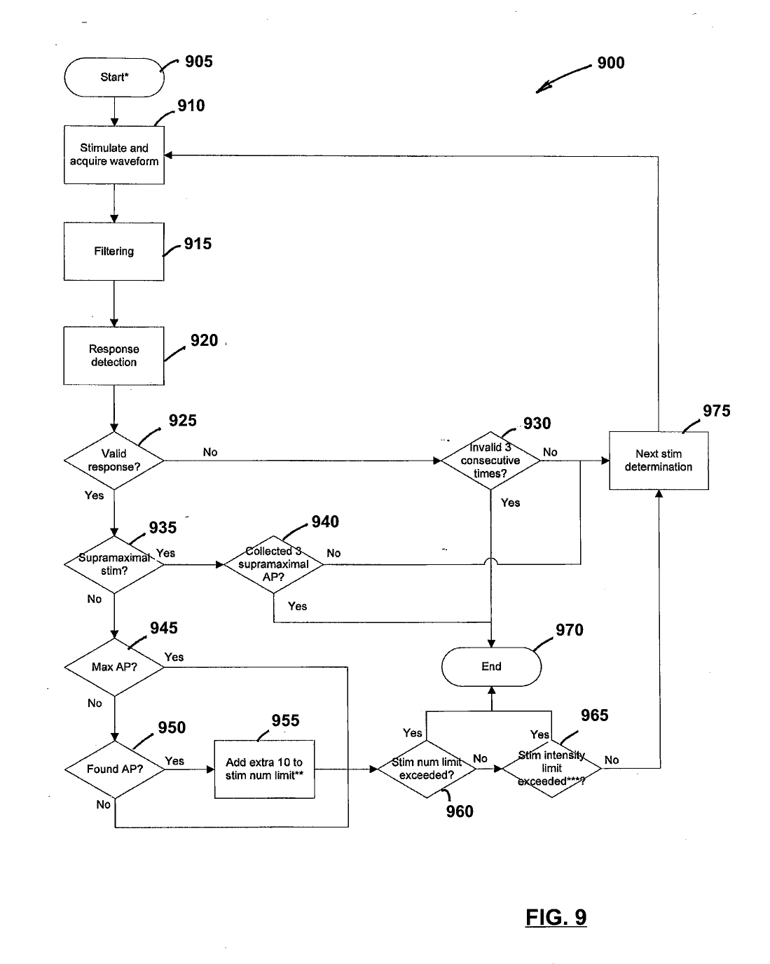

The testing process can be broken down into the following three consecutive stages: the search for a first response; the search for a maximal response; and the accumulation of maximal response measurement results. This three-stage approach is illustrated diagrammatically in FIG. 8.

During the first two stages of the testing process, stimulus intensity (I), calculated as current (C) multiplied by duration (D), changes by steps (increments). One increment may be, for example, a current increase or decrease of about 4 mA. The value of the increment may vary according to requirements. The stimulation duration remains unchanged if the changed current is not greater than a predetermined upper current limit (for example 50 mA) and is greater than 0 mA. If the changed current is greater than the predetermined current limit, or not greater than 0, the duration changes one step (increment), which may be, for example, 0.1 ms.

If it is desired to change the current intensity I1=C1×D1 to a new intensity I2=C2×D2, by n steps, then:

C2=C1±(n×4) and D2=D1 for C2>0 and C2<=50 ; or, if the new I2 would result in C2>50, or C2<0 then D2=D1±(duration increment) and C2=((C1×D1)/D2)±(n×4), for C2>0 and C2<=50, and D2>=lower duration limit and D2<=upper duration limit.

If the new I2 is not achievable within the current and duration limits, it is considered that the upper limit of the stimulus intensity has been met. The lower duration limit may be 0.1 seconds, for example. The upper duration limit may be 0.5 seconds, for example.

During the phase of searching for the first response, the stimulus intensity is increased by two steps for each new stimulus. Once the first response is detected, it is validated, and, if valid, the processor 114 turns to search for a maximal response. The processor 114 then causes the stimulus and data acquisition module to increase the stimulus intensity by two steps and then decrease it by one step alternately until processor 114 determines that a maximal response has been found. Once the maximal response is found, the stimulus intensity is set at a supra-maximal intensity and stays the same, i.e. 1.1 times the maximal intensity, until enough validated supra-maximal responses are accumulated.

Generally, the stimulus intensity is limited by having an upper duration limit of 0.5 ms and an upper current limit of 50 mA, for safety and comfort reasons. An example start intensity for a sensory nerve test may have a duration of 0.1 ms and a current of 10 mA. An example motor test may start with a duration of 0.2 ms and a current of 8 mA. As the intensity is increased during the testing (in search of a first response and then in search of a maximal evoked response), the duration of the stimulus may be increased in increments of 0.1 ms and the current may be increased in 4 mA increments. The frequency of the stimulus is preferably about 1 Hz, which results in a stimulus being provided to the body part about every second. This means that the analysis of the evoked response waveform and the determination of the next required stimulus intensity must be performed in much less than 1 second.

For median motor nerve testing, the stimulus point may be at the wrist and the recording point may be at the abductor pollicis brevis (APB). For the median sensory test, the stimulus point may be at the wrist and the recording point may be on the fourth digit. For the ulnar motor test, the stimulus point may be at the wrist and the recording point may be at the abductor digiti minimi (ADM). For the ulnar sensory test, the stimulus point may be at the wrist and the recording point may be at the fourth digit.

It should be noted that, while the sensing electrodes are generally described herein as being distally positioned when the stimulus unit 130 or 500 is in use, and the stimulation electrodes are described as being more proximally positioned, these positions represent nerve conduction testing in an antidromic orientation. It should be understood, however, that the relative functions of the sensing and stimulating electrodes may be reversed to an orthodromic orientation. When using stimulus unit 130 in an orthodromic orientation, the stimulus may be applied at the fingers and/or thenar and/or hypothenar areas and the evoked response sensing may occur at the wrist, for example.

For both the motor and sensory nerve tests, the response waveform is band pass filtered to eliminate frequencies outside of about 5 to 2000 Hz. For evoked CMAP responses, an amplitude threshold of about 0.5 mv for upper limb nerves (median and ulnar nerves) and 0.1 mv for lower limb nerves (tibial and peroneal nerves), is used together with an onset latency threshold of about 2 ms. For evoked SNAP responses, an amplitude threshold of about 2 μv is used, together with an onset latency threshold of about 2 ms for the median nerve and 1.5 ms for the ulnar nerve. These thresholds are used to restrict the focus of the analysis to only those parts of the evoked response waveform that are of interest for diagnostic purposes.

The test will be stopped when a predetermined number of satisfied traces is acquired. For example, six to ten traces may be considered to be sufficient. These accepted traces are saved in memory 112 in an appropriately sized stack.

The number of stimuli to be provided during a test is limited to a predetermined number, depending on whether a response is detected. The stimulus number limit is set to a first limit, say 20, if there is no response detected. The stimulus number limit is extended to a second limit, say 30, if one or more valid responses are found. When the stimulus intensity exceeds its limit (0.5 ms or 50 mA or both) or the number of stimuli reaches its limit (20 or 30), the test procedure is stopped, even if not enough satisfied responses are acquired.

For the median and ulnar nerves, each trace has about a 12 ms length of signal sampled at a 20 KHz sampling frequency, resulting in about 240 samples. For one test using 10 traces, for example, 2400 samples will be kept in memory. For the tibial, sural and peroneal nerves, the length of signal is 60 ms with a 20 kHz sampling frequency, i.e., 1200 samples. For one test (10 traces) 12000 samples will be kept in memory.

One task of the processor 114 is to determine the latency of the maximal action potential, which is defined as the duration between the onset of stimulus and the onset of the maximal action potential. To locate the onset of an AP, the peak of the AP is determined first. The AP onset is located after a negative peak of the response and is usually expected to occur after a latency threshold as described above.

After having found an AP, processor 114 analyzes the waveform (from the sample points) and starts to look for the onset of the AP. To do that, a range of 10% of peak amplitude of the AP above and below the base line is set, within which the AP onset is searched, as illustrated for a CMAP in FIG. 7. The start point is the intersection of the upper (+10%) line and the trace. Processor 114 searches backwardly from the start point. The AP onset is defined as the first point where the slope is lower than a predefined threshold, which is usually the base line.

A positive peak sometimes occurs before the peak of the AP (referred as initial positivity, for CMAP). To be detected, its amplitude needs to procedure. Its existence will be displayed to the user when the test is finished as a warning, which may trigger a re-test or manual invention.

Each time a response is detected, it is verified using historical information by the following rule: a stronger stimulus should elicit a stronger or equal response; and a weaker stimulus should evoke a weaker, equal or no response. Once an invalid response is determined, it is discarded and the stimulus is repeated up to 3 times until it finds a valid one. If it fails to evoke a valid response with the same stimulus after 3 times, and if there is/are valid response(s), the test stops, sending out the results with a warning as to the reliability of the results. On the other hand, if there is no valid response, the current one is discarded and the test continues with a stimulus intensity two steps higher.

Three parameters are used to determine and validate the detection of a maximal action potential (MaxAP): the amplitude, area and the onset latency of the response. The amplitude of the response is the difference between the peak amplitude of the response and the amplitude at onset. The area of the response is the region between the response curve and the base line from onset to the peak of the AP (see the shadowed area under the response curve of FIG. 7). The onset latency of the response is the elapsed time from simulus to onset.

Usually before a nerve or muscle reaches its maximal response potential (MaxAP), as the stimulus intensity (SI) increases, the response becomes stronger: amplitude and area increase and onset latency decreases or stays the same. The stimulus intensity that elicits a MaxAP is referred as the maximal stimulus intensity and the supra-maximal stimulus intensity is determined as a proportion of the maximal intensity, such as 110%, for example.

After the first valid response is detected, processor 114 dynamically averages the three latest waveforms and measures the response of the averaged waveform. When the averaged responses do not substantially change for three consecutive averaged waveforms, i.e. the changes of Amplitude and Area are less than 20%, and onset latency varies within 0.2 ms, they are defined as MaxAP. The stimulus intensity that resulted in the highest averaged response of these three consecutive averaged responses is determined to be the maximal stimulus intensity.

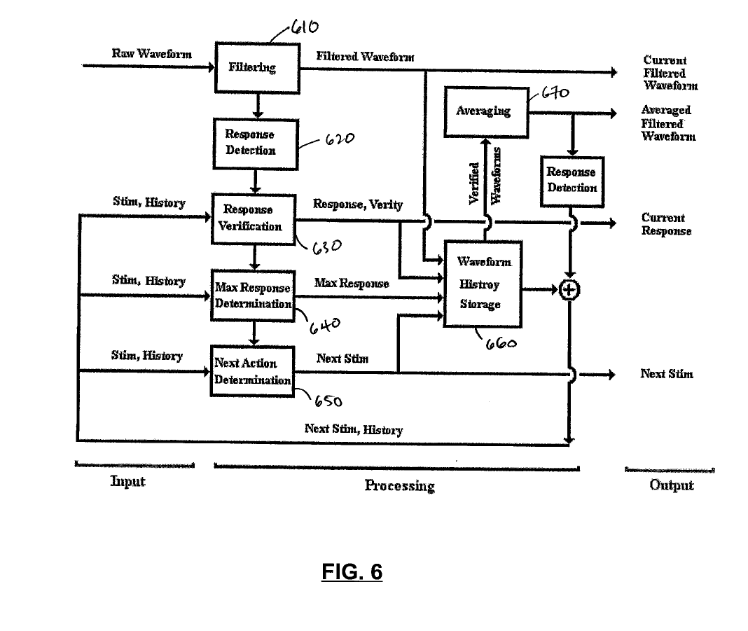

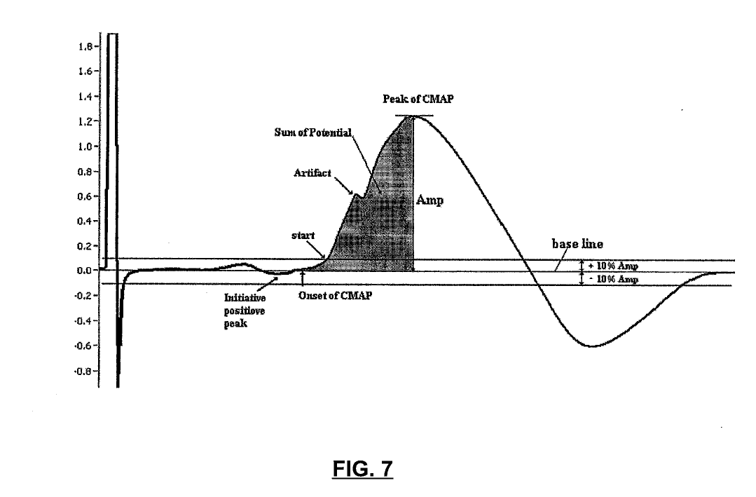

Referring now to FIG. 6, an example of the data flow, including inputs, outputs and processing, of system 100, is described in further detail. The main input received by system 100 is the raw waveform detected by the sensing electrodes on stimulus unit 130 (or 500). The raw waveform (610) is filtered and used to determine the occurrence of certain events (i.e. response detection), and is providing as output to a display on user interface 116, showing the current filtered waveform. The filtered waveform is also supplied to a response detection process 620, a response verification process 630, a maximal response determination process 640 and next action determination process 650. The filtered waveform is also supplied to memory 112 for storage within a waveform history storage module 660.

Filtered waveforms that indicate the existence of a response are subjected to the response verification process 630 to determine whether the response is as expected. The output of the response verification process 630 is stored in memory 112 in the waveform history storage module 660 and is provided to user interface 116 for display as the current response. Verified responses are provided to the maximal response determination process 640, which determines whether the verified response is a maximal response. The output of the maximal response determination process 640 is stored in the waveform history storage module 660 and provided to the next action determination process 650 to determine whether further stimulus is required and, if so, at what intensity. The output of the next action determination process 650 is stored in the waveform history storage 660 and provided to the stimulus and data acquisition module 120 for generation of appropriate stimulus currents to stimulus unit 130 (or 500).

An averaging process 670 is performed (as described below) on waveforms for which a response has been verified and the average filtered waveforms are provided as an output to user interface 116 and to processor 114 for use in determining whether a maximal response potential has been achieved.

The response verification, maximal response determination and next action determination processes (630, 640 and 650) receive the previous stimulus and history information as inputs in performing their respective functions. Further input and/or outputs may be used in the various processes described, specifically including inputs from the user of system 100 supplied by user interface 116 and various output displays and/or messages to the user to enable some level of user monitoring and control of the testing procedure, as desired.

For the exemplary data flow illustrated in FIG. 6, the inputs and outputs are summarized as follows.

Input:

Stimulus —response waveform.
Stimulus intensity and duration for the next stimulus to be delivered.
   Output:
Detected CMAP or SNAP onset latency and peak amplitude (indicated by a cursor position).
Intensity and duration of the next stimulus if the test is not finished.
Final results and possible message if the test is finished successfully.
Trouble shooting message if the test is terminated abnormally.

The method of determining an operable stimulus intensity described herein with reference to the drawings can be summarized as follows. As a starting point, a maximum stimulus pulse duration of 0.5 milliseconds is used, and a current limit of 50 milliamps is set. (The intensity is determined as the product of the duration and current). Starting at about 8 milliamps (two steps), the stimulus current is increased in steps of 8 milliamps (two steps) until the first stimulus response is detected. Immediately following detection of the first response, the current is decreased by about 4 milliamps (one step) in order to validate the response. If a response is then detected, which is less than the previously detected response, the first response is considered to be validated, given the relatively linear proportional relationship of the stimulus and response, at least at stimulus intensities less than the maximal stimulus intensity. If a response is not validated in the manner described, it is disregarded and the stimulus is repeated a certain number of times, say three or so.

Following validation of the response using the slightly lower current, a current increase of 8 milliamps (two steps) is again applied. The response to this higher current stimulus is also validated by checking whether the amplitude of the response is greater than that of the previously detected response. The stimulus current is increased (by 8 milliamps) and decreased (by 4 milliamps) alternately until the stimulus current reaches the limit of 50 milliamps, at which point the duration is increased by 0.1 milliseconds and the current level is reset to its initial level of 8 milliamps, and the process is repeated until the maximal stimulus intensity is found.

In order to determine the maximal stimulus intensity of a patient, the three consecutive averaged responses are used to form a moving average of three consecutive sets of three responses. In this way, five consecutive responses are used to form a moving average based on three averages. Other embodiments may employ a greater or lesser number of averages for comparison and responses used to form each averaged response. For each of the three averages, the area of the averaged response curve between onset time and the maximum amplitude of the curve is determined. If the area and amplitude of all three moving averages are within, say, 20% and the difference in onset time is at most, say, 0.2 milliseconds, the stimulus intensity for the average response having the highest response intensity is determined to be the maximum stimulus intensity. This intensity is then used to calculate an operating stimulus intensity of, say, 110% of the determined maximum stimulus intensity. Alternatively, a higher supra-maximal stimulus intensity, such as 120%, for example, may be used.

Referring in particular to FIG. 9, there is shown a method 900 for automatic nerve stimulus. Method 900 begins at step 905, with the setting of initial duration and current values for the first stimulus to be administered to the body part. At step 910, the stimulus is provided from stimulus unit 130 in response to output from stimulus and data acquisition module 120 and the resulting waveform is captured (as sensed by stimulus unit 130). At step 915, stimulus and data acquisition module 120 filters the captured waveform. At step 920, the filtered waveform is provided to processor 114, which attempts to detect an evoked response in the waveform by comparing it against predetermined amplitude and onset latency thresholds.

At step 925, processor 114 checks whether the response is “valid”. This process is not an assessment of whether a response was detected, but rather an assessment of whether the waveform that was received corresponds to a waveform that was expected to be received. For example, if no response was detected previously and the present waveform does not indicate that a response has yet been detected, this is considered to be a valid response as it is consistent with expectations derived from previous waveforms. On the other hand, if the peak amplitude of a previously detected response is higher than the peak amplitude of the presently detected response, but the previous stimulus intensity was lower, this is contrary to expectations and is considered anomalous and not “valid”. If an invalid response has been detected for three consecutive stimuli of the same intensity, at step 930, the testing procedure is ended, at step 970, and a message regarding the cause of the premature ending of the test is displayed on user interface 116.

If the response is determined to be valid, then at step 935, processor 114 checks whether the response is the result of a supra-maximal stimulus. If so, at step 940, processor 114 check whether at least three such supra-maximal stimulus responses have been received. If further supra-maximal stimulus responses are required for testing purposes, processor 114 stores the present response and proceeds to determine the next stimulus intensity at 975 (which for a supra-maximal stimulus response, will be the same supra-maximal stimulus intensity). If enough supra-maximal stimulus responses have been received at step 940, the testing procedure is ended at step 970 and is considered to have achieved the testing objective.

If the valid response is not to a supra-maximal stimulation, then at step 945, processor 114 performs calculations to determine whether the received waveform corresponds to a maximal response. If so, processor 114 proceeds to check whether the stimulation number limit, which may be, say 20, has been exceeded, at step 960 and, if not, the next stimulus intensity is determined at step 975. If, at step 945, the response is not considered to be a maximal response, then at step 950, the processor 114 checks whether the waveform indicates that a response was detected. If, at step 950, a response was found, the stimulation number limit is increased, by say 10, to its upper limit (or a second limit), at step 955. Otherwise, the stimulation number limit remains the same and it is checked at step 960 to determine whether the number of stimulations has exceeded the limit.

If the stimulation number limit has not been exceeded, processor 114 checks whether the stimulation intensity limit (as previously described) is exceeded by the proposed next stimulation intensity determined at step 975 and, if so, the test is ended at step 970. If, at step 960, the stimulation number limit has been exceeded, the test is ended at step 970, and a message is displayed at user interface 116 to that effect.

Exemplary embodiments of the invention are described herein, with reference to the accompanying drawings. It should be understood that various modifications or enhancements may be made to the described embodiments without departing from the spirit and scope of the invention, and all such modifications and enhancements are embraced by the spirit and scope of the invention.

Glossary

| Acronym or | |
|---|---|
| Abbreviation | Description |
| MaxAP | Maximal Action Potential |
| AP | Action Potential |
| CMAP | Compound Muscle Action Potential |
| SNAP | Sensory Nerve Action Potential |
| mV | Milli volt |
| ms | Millisecond |
| mA | Milliamp |

1. A method of determining an operable stimulus intensity for nerve conduction testing, comprising:
repeatedly stimulating a body portion adjacent a nerve at an increasing stimulus intensity;
detecting a response potential in response to each stimulation of the body portion;
determining a plurality of averaged responses based on the detected response potentials, each averaged response being an average of a set of at least two consecutive response potentials, each set of response potentials having at least one response potential not in another set;
determining at least two parameters of each averaged response;
determining that a maximal stimulus intensity has been reached when the respective at least two parameters of at least two averaged responses are within a predetermined range; and
determining the operable stimulus intensity as a predetermined proportion of the maximal stimulus intensity.

2. The method of claim 1, wherein the predetermined proportion is about 110%.

3. The method of claim 1, wherein the predetermined range is a percentage range of 0 to 20%.

4. The method of claim 1, wherein the at least two averaged responses comprise three averaged responses.

5. The method of claim 1, wherein the at least two averaged responses are consecutive averaged responses.

6. The method of claim 1, wherein the at least two parameters are selected from the group consisting of: onset time, peak amplitude and the area of the response potential between peak amplitude and onset.

7. The method of claim 1, wherein each averaged response comprises an average of three consecutive response potentials.

8. The method of claim 1, wherein the steps of stimulating and detecting comprise:
a) determining a stimulus intensity at which to provide a stimulus to the body portion;
b) stimulating the body portion with the stimulus at the stimulus intensity; and
c) detecting the response potential of the body portion in response to the stimulus.

9. The method of claim 8, wherein the steps of stimulating and detecting further comprise:
d) reducing the stimulus intensity by a first predetermined amount and once repeating steps b) and c);
e) verifying that the reduced stimulus intensity corresponds to detecting a reduced response potential in response to the reduced stimulus intensity;
f) increasing the stimulus intensity by a second predetermined amount larger than the first predetermined amount and repeating steps b) and c); and
g) repeating steps d), e), and f) until the detected response potential is determined to be a maximal response potential.

10. The method of claim 9, wherein step f) further comprises verifying that the increased stimulus intensity corresponds to detecting an increased response potential in response to the increased stimulus intensity.

11. The method of claim 1, wherein the body portion is at least one of a hand and wrist.

12. The method of claim 11, wherein the method further comprises attaching a stimulus unit to the hand and wrist, the stimulus unit comprising at least two stimulus electrodes for providing stimulus to the body portion and at least two sensing electrodes for sensing the response potential.

13. The method of claim 11, wherein the nerve is at least one of a median nerve and an ulnar nerve.

14. The method of claim 12, wherein the method further comprises measuring a separation of the at least two stimulus electrodes and the at least two sensing electrodes, following the attaching.

15. The method of claim 14, wherein the measuring is performed using a distance measurement member coupled to a part of the stimulus unit.

16. The method of claim 15, wherein the distance measurement member comprises an elongate strip having indicia for indicating the separation.

17. The method of claim 1, wherein the body portion is at least one of a leg, ankle and foot.

18. The method of claim 17, wherein the method further comprises attaching a stimulus unit to the leg and ankle, the stimulus unit comprising at least two stimulus electrodes for providing stimulus to the body portion and at least two sensing electrodes for sensing the response potential.

19. The method of claim 17, wherein the nerve is the sural nerve.

20. The method of claim 18, wherein the method further comprises measuring a separation of the at least two stimulus electrodes and the at least two sensing electrodes, following the attaching.

21. The method of claim 20, wherein the measuring is performed using a distance measurement member coupled to a part of the stimulus unit.

22. The method of claim 21, wherein the distance measurement member comprises an elongate strip having indicia for indicating the separation.

23. A method of automatic nerve stimulus, comprising:
a) determining a stimulus intensity at which to provide a stimulus to a body portion adjacent a nerve;
b) stimulating the body portion with the stimulus at the stimulus intensity;
c) detecting a response potential of the body portion in response to the stimulus;
d) reducing the stimulus intensity by a first predetermined amount and repeating steps b) and c);
e) verifying that the reduced stimulus intensity corresponds to detecting a reduced response potential in response to the reduced stimulus intensity;
f) increasing the stimulus intensity by a second predetermined amount larger than the first predetermined amount and repeating steps b) and c); and
g) repeating steps d), e), and f until the detected response potential is determined to be a maximal response potential.

24. The method of claim 23, wherein step f) further comprises verifying that the increased stimulus intensity corresponds to detecting an increased response potential in response to the increased stimulus intensity.

25. Computer readable storage storing computer program instructions which, when executed by a computerized testing apparatus, cause the computerized testing apparatus to perform the method of:
repeatedly stimulating a body portion adjacent a nerve at an increasing stimulus intensity;
detecting a response potential in response to each stimulation of the body portion;
determining a plurality of averaged responses based on the detected response potentials, each averaged response being an average of a set of at least two consecutive response potentials, each set of response potentials having at least one response potential not in another set;
determining at least two parameters of each averaged response;
determining that a maximal stimulus intensity has been reached when the respective at least two parameters of at least two averaged responses are within a predetermined range; and
determining the operable stimulus intensity as a predetermined proportion of the maximal stimulus intensity.

26. The computer readable storage of claim 25, wherein the steps of stimulating and detecting comprise:
a) determining a stimulus intensity at which to provide a stimulus to the body portion;
b) stimulating the body portion with the stimulus at the stimulus intensity; and
c) detecting the response potential of the body portion in response to the stimulus.

27. The computer readable storage of claim 26, wherein the steps of stimulating and detecting further comprise:
d) reducing the stimulus intensity by a first predetermined amount and once repeating steps b) and c);
e) verifying that the reduced stimulus intensity corresponds to detecting a reduced response potential in response to the reduced stimulus intensity;
f) increasing the stimulus intensity by a second predetermined amount larger than the first predetermined amount and repeating steps b) and c); and
g) repeating steps d), e), and f until the detected response potential is determined to be a maximal response potential.

28. A system for determining an operable stimulus intensity for nerve conduction testing, comprising:
a stimulator for stimulating a body portion adjacent a nerve and detecting a response to stimulation of the body portion;
a control module for controlling the stimulator; and
memory storing program instructions and accessible by the control module, wherein, when the program instructions are executed by the control module, the control module and stimulator are caused to:
repeatedly stimulate the body portion at an increasing stimulus intensity;
detect a response potential in response to each stimulation of the body portion;
determine a plurality of averaged responses based on the detected response potentials, each averaged response being an average of a set of at least two consecutive response potentials, each set of response potentials having at least one response potential not in another set;
determine at least two parameters of each averaged response;
determine that a maximal stimulus intensity has been reached when the respective at least two parameters of at least two averaged responses are within a predetermined range; and
determine the operable stimulus intensity as a predetermined proportion of the maximal stimulus intensity.

29. The system of claim 28, wherein the predetermined proportion is about 110%.

30. The system of claim 28, wherein the predetermined range is a percentage range of 0 to 20%.

31. The system of claim 28, wherein the at least two averaged responses comprise three averaged responses.

32. The system of claim 28, wherein the at least two averaged responses are consecutive averaged responses.

33. The system of claim 28, wherein the at least two parameters are selected from the group consisting of: onset time, peak amplitude and the area of the response potential between peak amplitude and onset.

34. The system of claim 28, wherein each averaged response comprises an average of three consecutive response potentials.

35. The system of claim 28, wherein the stimulating and detecting comprise:
a) determining a stimulus intensity at which to provide a stimulus to a body portion adjacent a nerve;
b) stimulating the body portion with the stimulus at the stimulus intensity; and
c) detecting a response potential of the body portion in response to the stimulus.

36. The system of claim 35, wherein the stimulating and detecting further comprise:
d) reducing the stimulus intensity by a first predetermined amount and repeating steps b) and c);
e) verifying that the reduced stimulus intensity corresponds to detecting a reduced response potential in response to the reduced stimulus intensity;
f) increasing the stimulus intensity by a second predetermined amount larger than the first predetermined amount and repeating steps b) and c); and
g) repeating steps d), e), and f until the detected response potential is determined to be a maximal response potential.

37. The system of claim 36, wherein step f further comprises verifying that the increased stimulus intensity corresponds to detecting an increased response potential in response to the increased stimulus intensity.

38. The system of claim 28, wherein the body portion is at least one of a hand and wrist.

39. The system of claim 28, wherein the nerve is at least one of a median nerve and an ulnar nerve.

40. The system of claim 28, further comprising a distance measurement member coupled to the stimulator for measuring a separation of stimulus electrodes and sensing electrodes on the stimulator.

41. The system of claim 28, wherein the body portion is at least one of a leg, ankle and foot.

42. The system of claim 28, wherein the nerve is a sural nerve.

43. A system for automatic nerve stimulus, comprising:
a stimulator for stimulating a body portion adjacent a nerve and detecting a response to stimulation of the body part;
a control module for controlling the stimulator; and
memory storing program instructions and accessible by the control module, wherein, when the program instructions are executed by the control module, the control module and stimulator are caused to:
a) determine a stimulus intensity at which to provide a stimulus to the body portion;
b) stimulate the body portion with the stimulus at the stimulus intensity;
c) detect a response potential of the body portion in response to the stimulus;
d) reduce the stimulus intensity by a first predetermined amount and repeat steps b) and c);
e) verify that the reduced stimulus intensity corresponds to detecting a reduced response potential in response to the reduced stimulus intensity;
f) increase the stimulus intensity by a second predetermined amount larger than the first predetermined amount and repeat steps b) and c); and
g) repeat steps d), e), and f until the detected response potential is determined to be a maximal response potential.

44. The system of claim 43, wherein step f further comprises verifying that the increased stimulus intensity corresponds to detecting an increased response potential in response to the increased stimulus intensity.
